SEQUENCE LISTING (2 sequences)

SEQ ID NO: 1 - DNA, 26 bp, Artificial
  Synthetic sequence
  misc_feature (18)..(26): n is a, c, g, or t
gtttcccagtcacgatcnnnnnnnnn

SEQ ID NO: 2 - DNA, 17 bp, Artificial
  Synthetic sequence
gtttcccagtcacgatc

(19) United States
(12) Patent Application Publication
(10) Pub. No.: US 2007/0218478 A1
(43) Pub. Date: Sep. 20, 2007

(54) Methods for fragmentation and analysis of nucleic acid

(75) Inventors: Qing Bai, Santa Clara, CA (US); Susana Salceda, San Jose, CA (US); Kai Wu, Mountain View, CA (US); Thong Nguyen, San Jose, CA (US); Charles G. Miyada, San Jose, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/612,454

(22) Filed: Dec. 18, 2006

Related U.S. Application Data
(60) Provisional application No. 60/750,940, filed on Dec. 16, 2005.
(60) Provisional application No. 60/753,281, filed on Dec. 21, 2005.
(60) Provisional application No. 60/784,269, filed on Mar. 20, 2006.

(51) Int. Cl. C12Q 1/68; C12P 19/34

(52) U.S. Cl. 435/6; 435/91.2

(57) ABSTRACT

Methods for fragmenting and labeling DNA in a single reaction volume and incubation step using a uracil DNA glycosylase, an apurinic/apyrimidinic endonuclease, and a terminal transferase are disclosed. In a preferred embodiment the UDG, AP and TdT activities are first mixed together to form an enzyme mixture and then the enzyme mixture is mixed with the uracil containing DNA. The fragmentation and labeling reactions thus take place simultaneously as part of the same reaction. The methods may be used in a variety of applications where fragmenting and end-labeling single or double stranded DNA is desired.

### RELATED APPLICATIONS

This application claims the benefit of US Provisional Application Nos. 60/750,940 filed Dec. 16, 2005, 60/753,281 filed Dec. 21, 2005 and 60/784,269 filed Mar. 20, 2006, the entire disclosures of which are incorporated herein by reference for all purposes.

### FIELD OF THE INVENTION

The invention is related to methods, assays and reagent kits for fragmenting and labeling nucleic acids and for identifying regions of DNA bound by DNA binding proteins.

### BACKGROUND OF THE INVENTION

Nucleic acid hybridization methods often benefit from fragmentation and labeling of the target nucleic acids prior to hybridization. The conventional method for fragmentation of DNA molecules utilizes DNase I to digest the DNA molecules, which is a controlled enzymatic process with no specific sequence preference. The products of DNase I digestion are fragments with 3′—OH termini ready for terminal labeling by terminal transferase (TdT). The process of DNase I digestion is difficult to modulate to avoid over or under digestion which produces fragments with less than desired length. There remains a need in the art for methods for reproducibly and efficiently fragmenting nucleic acids for hybridization to microarrays.

Chromatin immunoprecipitation assays have become an important method in the identification of binding sites for nucleic acid binding proteins, such as transcription factors. These methods have also been used to determine genomic areas of active transcription and for studies of chromatin structure.

### BRIEF DESCRIPTION OF THE DRAWINGS

FIG. 1 is a schematic of a method of generating an amplicon containing labeled single-stranded sense cDNA fragments from an RNA sample.

FIG. 2 is a schematic of a method of generating an amplicon containing labeled double-stranded cDNA fragments from an RNA sample.

FIG. 3 is a schematic for a method of performing chromatin immunoprecipitation with analysis on an array.

### SUMMARY OF THE INVENTION

Methods for fragmenting and labeling DNA in a single reaction volume are provided. In general reaction conditions that are compatible with UDG, APE 1 and TdT are disclosed. Kits with mixtures of UDG, APE 1 and TdT are also disclosed.

In preferred embodiments the fragmentation and labeling method is combined with nucleic acid amplification methods to analyze nucleic acid samples. The fragmented and labeled samples are preferably hybridized to an array of nucleic acid probes to determine expression levels of RNA in complex nucleic acid mixtures.

In another embodiment the methods of fragmenting and labeling are combined with methods for performing chromatin immunoprecipitation. The amplified nucleic acid is hybridized to an array for analysis and identification of genomic regions bound to proteins of interest.

The above implementations are not necessarily inclusive or exclusive of each other and may be combined in any manner that is non-conflicting and otherwise possible, whether they are presented in association with a same, or a different, aspect of implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### DETAILED DESCRIPTION OF THE INVENTION

(A) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. All references to the function log default to e as the base (natural log) unless stated otherwise (such as log₁₀).

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No 6,300,070 and U.S. patent application Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 5,242,794, 5,494,810, 4,988,617 and in U.S. Ser. No. 09/854,317 and US Pub. No. 20030143599, each of which is incorporated herein by reference. In some embodiments DNA is amplified by multiplex locus-specific PCR. In a preferred embodiment the DNA is amplified using adaptor-ligation and single primer PCR. Other available methods of amplification, such as balanced PCR (Makrigiorgos, et al. (2002), Nat Biotechnol, Vol. 20, pp.936-9), may also be used.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al, Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Computer methods related to genotyping using high density microarray analysis may also be used in the present methods, see, for example, US Patent Pub. Nos. 20050250151, 20050244883, 20050108197, 20050079536 and 20050042654.

Related methods for preparing and analyzing nucleic acids on arrays are disclosed, for example, in US Patent Publication Nos. 20060134652, which discloses methods for fragmenting cDNA prepared from RNA using uracil incorporation, 20050106591 which discloses methods of preparing cDNA from RNA using random primers attached to an RNA polymerase promoter,

Additionally, the present invention may have preferred embodiments that include methods for providing genetic information over networks such as the Internet as shown in U.S. patent applications Ser. No. 10/063,559, 60/349,546, 60/376,003, 60/394,574, 60/403,381.

(B) Definitions

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An oligonucleotide or polynucleotide is a nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term fragment refers to a portion of a larger DNA polynucleotide or DNA. A polynucleotide, for example, can be broken up, or fragmented into, a plurality of fragments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

“Genome” designates or denotes the complete, single-copy set of genetic instructions for an organism as coded into the DNA of the organism. A genome may be multi-chromosomal such that the DNA is cellularly distributed among a plurality of individual chromosomes. For example, in human there are 22 pairs of chromosomes plus a gender associated XX or XY pair.

The term “chromosome” refers to the heredity-bearing gene carrier of a living cell which is derived from chromatin and which comprises DNA and protein components (especially histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A “chromosomal region” is a portion of a chromosome. The actual physical size or extent of any individual chromosomal region can vary greatly. The term “region” is not necessarily definitive of a particular one or more genes because a region need not take into specific account the particular coding segments (exons) of an individual gene.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991). Each of which is incorporated by reference in its entirety for all purposes.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. No. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as optical fibers, glass or any other appropriate substrate. (See U.S. Pat. Nos. 5,770,358, 5,789,162, 5,708,153, 6,040,193 and 5,800,992, which are hereby incorporated by reference in their entirety for all purposes.)

Preferred arrays are commercially available from Affymetrix under the brand name GENECHIP® and are directed to a variety of purposes, including genotyping and gene expression monitoring for a variety of eukaryotic and prokaryotic species. (See Affymetrix Inc., Santa Clara and their website at affymetrix.com.) Methods for preparing sample for hybridization to an array and conditions for hybridization are disclosed in the manuals provided with the arrays, for example, for expression arrays the GENECHIP Expression Analysis Technical Manual (PN 701021 Rev. 5) provides detailed instructions for 3′ based assays and the GeneChip® Whole Transcript (WT) Sense Target Labeling Assay Manual (PN 701880 Rev. 2) provides whole transcript based assays. The GeneChip Mapping 100K Assay Manual (PN 701694 Rev. 3) provides detailed instructions for sample preparation, hybridization and analysis using genotyping arrays. Each of these manuals is incorporated herein by reference in its entirety.

(C) One Step Fragmentation and Labeling

Prior art methods of fragmenting and labeling cDNA included a first fragmentation step where UDG and APE 1 are used to fragment uracil containing cDNA and a second labeling step where the fragments are end labeled using TdT. The methods disclosed herein disclose methods for combining the fragmentation and labeling steps into a single incubation. The methods are particularly useful for automation as they eliminate liquid handling steps and reduce the overall time of incubations. In a preferred aspect uracil containing cDNA is synthesized and the uracil containing cDNA is fragmented by uracil DNA glycosylase (UDG) and an AP endonuclease such as APE 1. The fragments may be labeled in an end-labeling reaction with a terminal transferase. Terminal transferase (TdT) is a template independent polymerase that catalyzes the addition of deoxynucleotides to the 3′ hydroxyl terminus of DNA molecules. Protruding, recesses or blunt-ended double or single-stranded DNA molecules are substrates for TdT. Efficient incorporation by TdT requires the presence of the divalent cation Co²⁺.

When multiple enzymatic steps are combined into a single reaction it is beneficial to find reaction conditions that are tolerable for all of the enzymes. These conditions may not be optimal for any one of the enzymes or they may be selected to be optimal for one of the enzymes, but not for the others. When combining fragmentation and labeling, the enzymes may include a UDG, an AP endonuclease and a TdT in the same reaction. The source of the enzyme may also be considered when selecting reaction conditions. Enzymes that are structurally similar (same amino acid sequence) from different vendors may not perform identically. This may be due, for example, to different manufacturing or shipping conditions. Often enzyme may be purchased from a vendor with a buffer that is recommended by the manufacturer. For example, the UDG reaction buffer is 20 mM Tris-HCl, 1 mM dithiothreitol, and 1 mM EDTA, pH 8.0 at 25° C., the buffer for APE 1 is NEBuffer 4 which is 20 mM Tris-acetate, 50 mM potassium acetate, 10 mM magnesium acetate, and 1 mM dithiothreitol, pH 7.9 at 25° C. The buffer for TdT from NEB is NEBuffer 4 plus CoCl₂ which is 20 mM Tris-acetate, 50 mM potassium acetate, 10 mM magnesium acetate, 1 mM dithiothreitol, pH 7.9 at 25° C. and 0.25 mM CoCl₂. The buffer for Promega TdT is 100 mM cacodylate buffer (pH 6.8 at 25° C.), 1 mM CoCl₂, and 0.1 mM DTT. The buffer for Roche TdT is 200 mM potassium cacodylate, 25 mM Tris-HCl, 0.25 mg/ml BSA (pH 6.6 at 25° C.), and 5 mM CoCl₂. The buffer for Invitrogen TdT is 100 mM potassium cacodylate (pH 7.2), 2 mM CoCl₂, and 0.2 mM DTT.

The UDG enzyme is active over a broad pH range with an optimum pH of about 8.0. UDG does not require a divalent cation and is inhibited at high ion strength, for example, greater than about 200 mM.

Enzymes are provided from a vendor are provided in solution in a storage buffer. Human APE 1 is provided from NEB in 10 mM Tris-HCl, 50 mM NaCl, 1 mM DTT, 0.05 mM EDTA, 200 μg/ml BSA, 50% glycerol, pH 8.0 at 25° C. and stored at −20° C. UDG from NEB is in 10 mM Tris-HCl (pH 7.4), 50 mM KCl, 1 mM DTT, 0.1 mM EDTA, 200 μg/ml BSA, 50% glycerol and stored at −20° C. TdT from NEB is in 60 mM KPO4, 150 mM KCl, 1 mM 2-Mercaptoethanol, 0.5% TRITON X-100 and 50% glycerol at pH 7.2 at 25° C. In one embodiment of the present methods the enzymes are mixed together by adding 1 part APE 1, 1 part UDG and 8 parts TdT. As a result 80% of the buffer of the mixture is contributed by the TdT storage buffer.

In a preferred aspect the three enzymes, APE 1, UDG and TdT may be purchased from a single vendor. As a result of differences in manufacturing and formulation the same enzyme purchased from a different vendor may have slightly different activity and may perform optimally at different conditions. For example, different sources of TdT were tested in the present methods with varying results. In a preferred aspect the fragmentation and labeling reaction is optimized to work with APE 1, UDG and TdT from NEB. NEB TdT was tested with varying concentrations of CoCl₂. The following concentrations were tested: 0.5 mM, 1 mM, 2 mM and 4 mM CoCl₂ in NEB buffer 4.

In a preferred embodiment the fragmentation and labeling reaction includes about 5 μg Single-Stranded DNA, 5 μL10X NEBuffer 4, 2 μL 25 mM CoCl₂, 1 μL 1,000 μL APE 1 (NEB), 1 μL 10U/μL UDG (NEB), 1μL 5 mM DLR and either 4 μL 30 U/μL TdT (Promega) or 8 μL 20 U/μL TdT (NEB) and nuclease-free water up to 50 μL. The reaction is mixed, quick spun and incubated at 37° C. for either 60 or 90 minutes then incubated at 70° C. for 10 minutes followed by incubation at 4° C. for 2 minutes.

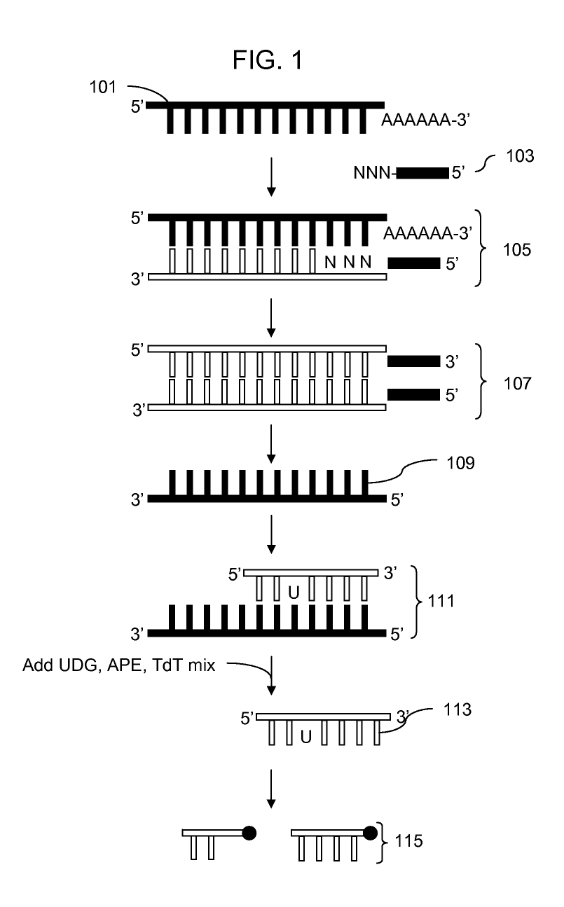

FIG. 1 shows a schematic of a preferred embodiment. A sample containing RNA (101) is reverse transcribed using T7-(N)₆ primers (103) to generate an RNA:DNA hybrid (105). Second strand cDNA synthesis generates a double-stranded cDNA with a T7 promoter (107). The double-stranded cDNA is used as template in an in vitro transcription reaction resulting in the production of antisense cRNA (109) which is preferably unlabeled. The antisense cRNA is used as template in a reverse transcription reaction primed by random primers and in the presence of a mixture of dGTP, dCTP, dTTP, dATP and dUTP, generating cDNA containing uracil in RNA:DNA hybrids (111). The cRNA may be removed or hydrolyzed, for example, by RNase H treatment, leaving single-stranded uracil containing cDNA (113). The cDNA (113) may be cleaned up and mixed with UDG, APE 1 and TdT under conditions where each of the 3 enzymes is active to generate labeled cDNA fragments (115). The cDNA fragments may be end labeled using TdT and DLR. In a particularly preferred embodiment the RNA sample (101) is total RNA that has been subjected to one or more steps for reduction of ribosomal RNA, for example, by treatment with RIBOMINUS from Invitrogen.

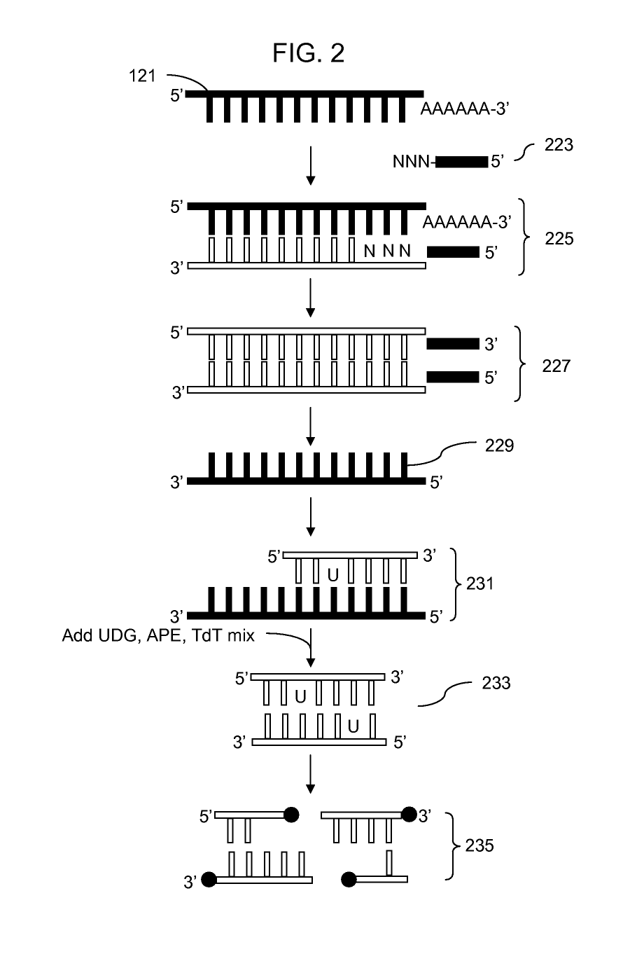

In another embodiment, shown in FIG. 2, sense and antisense cDNA is generated and double stranded cDNA is fragmented by an AP endonuclease. A sample containing RNA (221) is reverse transcribed using T7-(N)₆ primers (223) to generate an RNA:DNA hybrid (225). Second strand cDNA synthesis generates a double-stranded cDNA with a T7 promoter (227). The double-stranded cDNA is used as template in an in vitro transcription reaction resulting in the production of antisense cRNA (229) which is preferably unlabled. The antisense cRNA is used as template in a reverse transcription reaction primed by random primers and in the presence of a mixture of dGTP, dCTP, dTTP, dATP and dUTP, generating cDNA containing uracil in RNA:DNA hybrids (231). E. coli DNA polymerase and RNase H are added to generate second strand cDNA, resulting in double-stranded cDNA (233). Both strands of the ds-cDNA contain uracil. UDG and APE 1, or another AP endonuclease that cleaves double stranded DNA, and TdT are added to fragment the DNA and end label the fragments, generating labeled double stranded cDNA fragments (235). Fragmentation and labeling take place in the same reaction and under the same reaction conditions so they are essentially simultaneous. In preferred aspects E.coli DNA polymerase is used if the desired target is single stranded cDNA, because the enzyme is less prone to spurious copying of the original strand. Where the desired product is double-stranded target polymerases such as Klenow (exo-) may be preferred. Klenow is more prone to creating copies of the original strand.

Methods for using apurinic/apyrimidinic endonuclease for fragmentation and end-labeling of DNA molecules are disclosed. Single or double-stranded nucleic acid molecules may be fragmented and labeled. In a preferred embodiment DNA molecules that may be end-labeled according to the methods are nucleic acids that, once fragmented, have a free 3′ hydroxyl group. The DNA molecules can be any desired chemically and enzymatically synthesized nucleic acid, e.g., a nucleic acid produced in vivo by a cell or by in vitro amplification.

In a preferred embodiment an apurinic/apyrimidinic endonuclease is used to cleave an apyrimidinic site within a DNA molecule to yield a fragment with a certain range of length and a 3′—OH terminus. The 3′—OH terminus may be used for terminal labeling. In some embodiments the apurinic/apyrimidinic endonuclease generates a 3′-phosphate terminus and the phosphate is subsequently removed, for example, by adding phosphatase to the reaction, generating a 3—OH terminus conducive for subsequent terminal labeling. In a preferred embodiment, apurinic/apyrimidinic endonucleases which create a 3′—OH terminus that may be used include, endonuclease V, endonuclease VI, endonuclease VII, human endonuclease II, and the like. In the subject invention, apurinic/apyrimidinic endonucleases which create a 3′-phosphate terminus consist of, but are not limited to endonuclease III, endonuclease VIII, and the like. Any apurinic/apyrimidinic endonuclease involving hydrolytic based cleavage would be appropriate for use with the disclosed methods.

The fragmentation process employed in the subject method begins with creating cleavable fragments. The first step in creating these fragments is the incorporation of an exo-nucleotide (a nucleotide which is generally not found in the sample DNA molecule or nucleic acid) or the incorporation of normal nucleotides that are then converted to exo-nucleotides into a sample DNA molecule or sample nucleic acid. dUTP is an example of an exo-nucleotide because generally it is rarely or found naturally in DNA. Although the triphosphate form of dUTP is present in living organisms as a metabolic intermediate, it is rarely incorporated into DNA. When dUTP is accidentally incorporated into DNA, the resulting deoxyuridine is promptly removed in vivo by normal process, e.g., processes involving the enzyme UDG. Thus, deoxyuridine occurs rarely or never in natural DNA. It is recognized that some organisms may naturally incorporate deoxyuridine into DNA. See U.S. Pat. No. 5,035,996. Normal nucleotides can be converted into exo-nucleotides by converting neighboring pyrimidine or purine residues, i.e. converting neighboring pyrimidine residues in thymidine to create pyrimidines dimmers. See U.S. Pat. Nos. 5,035,996 and 5,683,896.

In a preferred embodiment the DNA to be fragmented is a product amplified from a nucleic acid sample isolated from a biological source. In a preferred embodiment the DNA to be fragmented is an amplification product resulting from amplification of an RNA sample isolated from one or more cells. In a particularly preferred embodiment RNA is isolated from a source, first strand cDNA is generated by reverse transcription with primers comprising a random 3′ sequence and a 5′ RNA polymerase promoter sequence, for example, random hexamer-T7 primers, the first strand cDNA is used to generate second strand cDNA resulting in dsDNA with an RNA polymerase promoter, and unlabeled cRNA is transcribed by IVT. The antisense RNA (cRNA) product is the output of the first cycle of amplification and is used as the starting template for a second cycle of amplification. In the second cycle first strand cDNA is synthesized using the cRNA as template for an extension reaction primed by random primers. During this second cycle of first strand cDNA synthesis dUTP is present and is incorporated into the cDNA. The cRNA may then be hydrolyzed, for example, by treatment with RNase H and the sense stranded cDNA can be cleaned-up. The cDNA may then be treated with UDG and APE 1 to fragment and then fragments may be end labeled using TdT and a labeled nucleotide such as Affymetrix′ DNA Labeling Reagent. The labeled cDNA may then be hybridized to an array.

In another aspect the second cycle of amplification includes an optional step of second strand cDNA synthesis and the products are double-stranded cDNA In the second round of cDNA synthesis uracil may be incorporated into the first strand cDNA or the second strand cDNA or both. For a detailed example see Example 3 below.

The amount of starting material may be, for example, about 10 or 100 to 500 ng of total RNA. In some aspects less than 10 ng total RNA may be used as starting material. If the total RNA is subjected to a complexity reduction step, for example, depletion of rRNA or globin mRNA or enrichment of mRNA, less RNA may be used as starting material. Preferably about 5 or 10 to 100 μg and more preferably about 20 μg of labeled target may be used for hybridization to one array. In some embodiments total RNA may be treated to remove selected sequences that may interfere with analysis, for example, ribosomal RNA (rRNA) may be removed prior to amplification. Many methods of removing rRNA are known to one of skill in the art, for example, see U.S. Pat. No. 6,613,516 which describes hybridization of oligonucleotides that are complementary to ribosomal RNA to the ribosomal RNA, optionally extending the oligonucleotides and cleaving the rRNA with RNaseH activity. Another method of depleting rRNA, or another RNA that is not of interest, that may be used is to incubate the total RNA with a solid support (for example, beads, membrane or resin) comprising oligonucleotides that are complementary to rRNA sequences to allow rRNA to bind to the solid support. The bound rRNA may then be separated from the remaining total RNA that is in solution. In another embodiment globin mRNAs may be removed or depleted. Globin mRNAs are present in very high amounts in RNA isolated from blood and can interfere with detection of other mRNAs. Globin mRNAs may be removed, for example, by depletion using a solid support that has globin complementary oligonucleotides associated or attached as described above for rRNA, by hybridization of blocking oligonucleotides to the globin mRNA, the blocking oligos may prevent amplification of globin mRNAs by blocking reverse transcription of the globin mRNAs, or the globin mRNA may be depleted by hybridization of globin complementary oligos, optionally extension of the oligos and cleavage of the mRNA with RNase H. In some embodiments the oligonucleotides used contain one or more modified nucleotides, for example, peptide nucleic acids (PNAs) or locked nucleic acids (LNAs). For additional description of these methods see, for example, U.S. Pat. No. 6,613,516 and U.S. patent application Ser. No. 10/684,205. When rRNA is depleted less of the final product may be hybridized to a single array, for example, in one embodiment without rRNA depletion 20 μg is hybridized to an array and with rRNA depletion 5 μg of the labeled, fragmented cDNA is hybridized to the array.

In a preferred embodiment dUTP is incorporated into the sample DNA molecule or sample nucleic acid. dUTP can be incorporated via a reverse transcription reaction, preferably a specific ratio of dTTP to dUTP is used. This ratio of dTTP to dUTP is selected to generate DNA fragments of a pre-determined size range. In one preferred embodiment the fragment lengths show a peak, for example on a bioanalyzer, centered around 40 to 70 bases with more than 50% of the fragments ranging from 20 and 200 bases in length. In a preferred embodiment of the invention, the reverse transcription reaction is run so that the total RNA is reverse transcribed with dNTPs at a final concentration of about 0.5 mM. See U.S. Pat. Nos. 5,035,996 and 5,683,896

Next, the sample DNA molecules or nucleic acids are processed in a reaction comprising DNA glycosylase to create an abasic site. DNA glycosylases release bases from DNA by cleaving the glycosidic bond between the deoxyribose of the DNA sugar-phosphate backbone and the base. DNA glycosylases are capable of releasing, including but not limited to, cytosine bases from ssDNA and dsDNA, thymine bases from ssDNA and dsDNA, and uracil bases from ssDNA or dsDNA. DNA glycosylases are base specific. Therefore, the appropriate DNA glycosylase is dependent upon which base was incorporated into the sample DNA molecule or sample nucleic acid. See U.S. Pat. No. 6,713,294.

In the preferred embodiment of the subject invention, UDG specifically recognizes uracil and removes it by hydrolyzing the N-Cl′ glycosylic bond linking the uracil base to the deoxyribose sugar. The loss of the uracil creates an abasic site (also known as an AP site or apurinic/apyrimidinic site) in the DNA. An abasic site is a major form of DNA damage resulting from the hydrolysis of the N-glycosylic bond between a 2-deoxyribose residue and a nitrogenous base. This site can be generated spontaneously or as described above, via UDG catalyzed hydrolysis See Marenstein et al. (2004) DNA Repair 3:527-533. Treatment of the sample DNA molecule or sample nucleic acid with alkaline solutions or enzymes, such as but not limited to apurinic/apyrimidinic endonucleases, will cause controlled breaks in the DNA at the abasic site. See U.S. Pat. No. 6,713,294. The abasic site can be cleaved by physical or enzymatic means. While high temperature or high pH induced hydrolysis can generate cleavage at abasic sites, the resulting 3′ termini of the cleavage may not be a substrate for labeling by TdT. An apurinic/apyrimidinic endonuclease can cleave the DNA molecule or nucleic acid at the site of the dU residue yielding fragments possessing a 3′—OH termini, thus allowing for subsequent terminal labeling. One such apurinic/apyrimidinic endonuclease is E. coli Endo IV which catalyzes the formation of single-strand breaks at apurinic and apyrimidinic sites within a double-stranded DNA to yield 3′—OH termini suitable for terminal labeling. E. coli Endo IV may also be used to remove 3′ blocking groups (e.g. 3′-phosphoglycolate and 3′-phosphate) from damaged ends of double-stranded DNA. See Levin, J. D., J. Biol. Chem., 263:8066-8071 (1988) and Ljungquist, et al., J. Biol. Chem., 252:2808-2814 (1977).

In preferred aspects the cRNA generated from the IVT reaction by the first cycle of the assay is random primed to generate single or double-stranded DNA containing uracil. The uracil base is specifically removed from the DNA by UDG and in the same reaction APE 1 cleaves the phosphodiester backbone where the base is missing, leaving a 3′ hydroxyl and a 5′ deoxyribose phosphate terminus. Also in the same reaction TdT catalyzes the addition of DLR to the the 3′ hydroxyl termini of the DNA fragments.

In a preferred embodiment the AP endonuclease is human APE 1 or a variant thereof. Human APE 1, unlike E. coli Endo IV, is capable of cleaving either single-stranded or double-stranded substrate at AP sites. APE 1 is also known as Hapl Apex, and Refl and can be utilized in conjugation with UDG to perform cleavage at dU incorporation sites in single-strand and double strand DNA. APE 1 is an enzyme of the base excision repair pathway which catalyzes endonucleolytic cleavage immediately 5′ to abasic sites. See Marenstein supra. Additional information about APE 1 may be found in Robson, C. N. and Hickson, D. I. (1991) Nucl. Acids Res., 19, 5519-5523, Vidal, A. E. (2001)EMBO J., 20,6530-6539, Demple, B. et al. (1991) Proc. Natl. Acad. Sci. USA, 88, 11450-11454, Barzilay, G. et al. (1995) Nucl. Acids Res., 23, 1544-1550, Barzilay, G. et al. (1995) Nature Struc. Biol., 2, 451-468, Wilson, D. M. III et al. (1995) J. Biol. Chem., 270, 16002-16007, Gorman, M. A. et al (1997) EMBO J., 16, 6548-6558, Xanthoudakis, S. et al. (1992) EMBO J., 11, 3323-3335, Walker, L. J. et al. (1993) Mol. Cell Biol., 13, 5370-5376, and Flaherty, D. M. (2001) Am. J. Respir. Cell. Mol. Biol., 25, 664-667, each of which is incorporated herein by reference in its entirety for all purposes.

APE 1 acts on both dsDNA and ssDNA. The catalytic efficiency of the cleavage of ssDNA is approximately 20-fold less than the activity against AP sites in dsDNA. Catalysis is Mg²⁺ dependent. Unlike the activity of APE 1 against AP sites in dsDNA, it does not display product inhibition when acting on an AP site in ssDNA. One unit of APE 1 is defined by the supplier (New England Biolabs) as the amount of enzyme required to cleave 20 pmol of a 34 mer oligonucleotide duplex containing a single AP site in a total reaction volume of 10 μl in 1 hour at 37° C.

The amount of dU incorporation may be regulated to determine the average length of fragments after UDG/APE 1 treatment. The ratio of dUTP to dTTP may be, for example, about 1 to 4, or about 1 to 5, 1 to 6, 1 to 10 or 1 to 20. One of skill in the art will appreciate that varying the ratio of dUTP to dTTP will result in variation of the amount of dUTP incorporated and result in variation in the average size of fragments. The higher the ratio of dUTP to dTTP the more uracil incorporated and the shorter the average size of the fragments. In a preferred embodiment the fragments are on average about 40 to 70 nucleotides in length, with more than 90% of the fragments being between 25 and 150 bases in length. In another embodiment the fragments are on average between 25 and 50, 40 and 70, 40 and 80, 50 and 100 or 30 to 150 bases or base pairs in length. Longer or shorter fragment sizes may also be achieved by varying the reaction conditions.

In some aspects kits are provided for obtaining amplified cDNA from RNA and fragmenting and labeling the cDNA for hybridization. In one aspect a fragmentation and labeling kit is provided. The kit may include, for example, cDNA fragmentation buffer, UDG, APE 1, TdT, TdT buffer, and a labeled nucleotide, for example, DLR. The components are preferably provided in a concentrated form, for example, buffers may be provided in the kit as 10X or 5X stocks. The UDG is preferably provided at about 10 U/μl and the APE 1 is preferably about 1000 U/μl. Higher concentrations of APE 1 are used for fragmentation of single-stranded cDNA target. In a preferred aspect the UDG, APE 1 and TdT may be provided in a single enzyme solution containing all three enzymes in an appropriate buffer solution.

In another aspect a kit for generating amplified sense strand cDNA from total RNA may be provided. The kit may include T7-(N)₆ primers at about 2.5 μg/μl, 5X first strand cDNA synthesis buffer, 100 mM DTT, 10 mM dNTP mix, RNase inhibitor (40 U/μl), MgCl₂ (1 M), a reverse transcriptase, such as SuperScript II, a DNA polymerase, such as DNA Pol 1, a random primer solution (3 μg/μl), RNase H (2 U/μl), water and a dNTP+dUTP mix. The kit may also include reagents for in vitro transcription including an NTP mix, 10×IVT buffer, IVT enzyme mix and IVT controls. The cDNA synthesis reagents may be organized in a first box as a first sub kit and the IVT reagents may be organized in a second box as a second sub kit. The first and second boxes may be packaged together in a third box.

When utilizing the above fragmentation method with APE 1 for single-stranded cleavage of cDNA, the RNA strand may be digested by either alkaline hydrolysis or enzymatic digestion. For example, the alkaline hydrolysis would occur in alkaline conditions at 55-75° C. for 20-40 minutes. Another example would be performing the enzymatic digestion with RNase H, or an enzyme with similar properties, at 27-47° C. for 20-60 minutes. The remaining DNA strand may then be purified before fragmentation. When utilizing the above method for double-stranded cleavage, a second strand DNA synthesis is performed and the double-stranded DNA is purified before fragmentation. The fragmentation of either single or double-stranded DNA is performed in the presence of UDG and APE 1 and appropriate buffering conditions for APE 1. The reaction is incubated at 27-47° C. for 1-2 hours. The enzymes are heat inactivated at about 93° C. for about 1 minute.

In a preferred embodiment fragmented DNA is labeled. Labeling in one embodiment is by end labeling, for example, labeling of 3′ hydroxyls using TdT. The fragments are incubated in a reaction with TdT, buffer, CoCl₂, and DNA labeling reagent (a biotinylated nucleotide analogue) or any other suitable label. The reaction may be incubated at 27-47° C. for about 1 hour. Preferably more than 80% of the fragments are labeled.

After the fragments have been end-labeled, the product of labeled DNA fragment may be hybridized to a microarray. Examples of microarrays that may be used for analysis are available from Affymetrix, Inc. and include, for example, the HG-U133A 2.0 array and more preferably a GENECHIP Exon Array such as the Human Exon 1.0 ST Array. Kits for whole transcript (WT) cDNA synthesis and amplification are available from Affymetrix (PN 900673). Kits for fragmentation and labeling are also available from Affymetrix (PN 900652). The fragmentation and labeling kit includes Affymetrix′ DNA labeling reagent (DLR) (biotin allonamide triphosphate) which has the structure shown below: 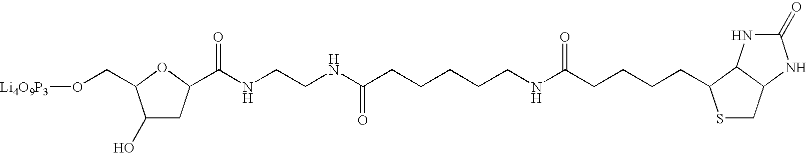
(D) Chromatin Immunoprecipitation and Array analysis methods:

The fragmentation and labeling methods disclosed above may be used in combination with genome analysis methods such as ChIP-on-chip assays or genotyping assays. In preferred embodiments methods for identification of genomic regions that are associated with one or more proteins are combined with the disclosed methods to provide methods for analysis of protein-DNA interactions. In general, nucleic acid is crosslinked to proteins that are in close proximity to the nucleic acid in the cell. The nucleic acid that is crosslinked to the protein is recovered by immunoprecipitation and identified by hybridization to an array of probes, the recovered nucleic acid or an amplification generated from the recovered nucleic acid is hybridized to the array to identify the bound regions by their presence in the recovered nucleic acid. The methods may be used to identify protein binding sites on nucleic acid.

Methods to identify specific regions of DNA bound to protein have been previously demonstrated. For example, Orlando et al., Methods: A companion to Methods in Enzymology, 11:205-214 (1997), demonstrated immunoprecipitation of in vivo cross-linked DNA associated with chromatin, amplification of the immunoprecipitated DNA and use of the amplified DNA as a probe to identify the genomic region associated with the protein. Orlando and Paro, Cell 75:1187-1198 (1993) also used PCR amplification of immunoprecipitated DNA to identify DNA binding sites for proteins. More recent studies include Ng et al. Genes & Dev. 16:806-819 (2002), Ren et al., Science: 290:2306-2309(2000); Cawley et al., Cell 116:499-509 (2004) and Bernstein et al., Cell 120:169-181 (2005).

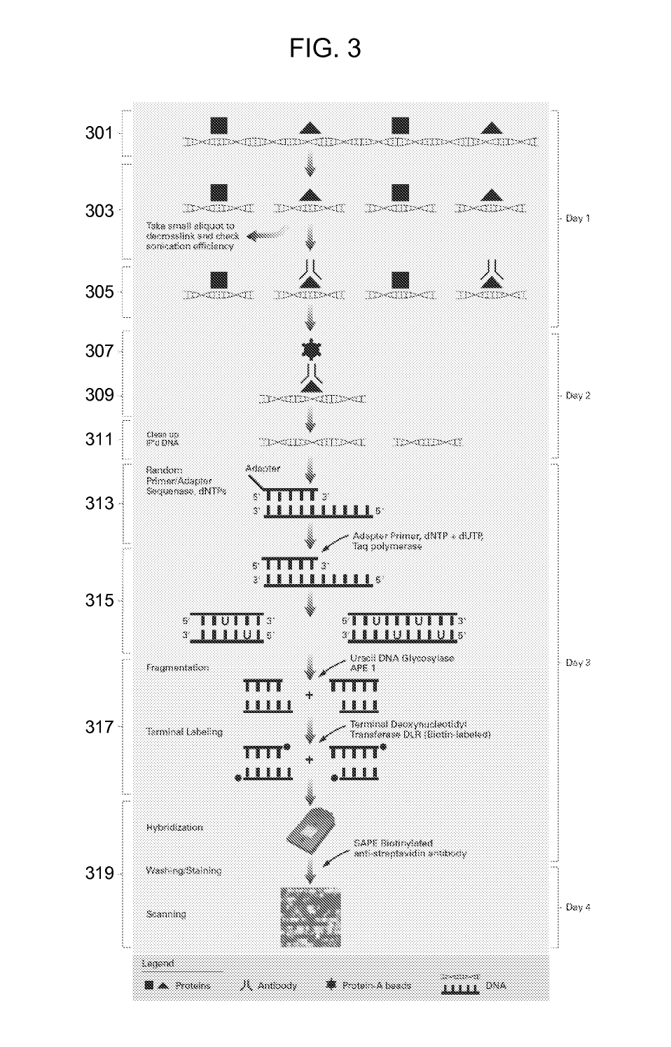

The general steps of the method are shown in FIG. 3. Cells are fixed to crosslink DNA to protein [301]. The cells are then sonicated to lyse the cells and shear chromatin [303]. The sample is incubated with one or more selected antibodies to allow complexes to form [305]. The antibodies are then coupled to protein-A beads [307] and the beads washed to purify the immunoprecipitated DNA [309]. The purified DNA is then recovered and cleaned [311] and amplified by extending a primer that has a 3′ random primer region and a 5′ constant adapter region [313] followed by PCR using a primer to the common adapter region and incorporation of dUTP [315]. The PCR products are then fragmented using uracil DNA glycosylase and APE1 and terminal labeled using TdT and a biotin labeled nucleotide [317]. The labeled sample is hybridized to an array. The array is washed, stained and scanned to generate a pattern that is indicative of the hybridization of the sample to the probes of the array [319].

In preferred aspects the binding sites are binding sites for transcription factors and the methods allow identification of areas of active transcription in genomic DNA. The methods may also be used to assess modifications of genome structure resulting from histone binding.

The Affymetrix Chromatin Immunoprecipitation (ChIP) Assay is designed to generate double stranded labeled DNA targets which interrogate sites of protein-DNA interactions or chromatin modifications on a genome-wide scale. In preferred aspects the methods may be used with Affymetrix GeneChip® Tiling Arrays for ChIP on chip studies in order to study epigenetic phenomena such as transcription factor binding sites, histone protein modifications, and DNA methylation.

In general the term tiling array refers to an array that comprises probes that are spaced evenly over a target region. The probes of the array may be spaced, for example, so that the gap between two probes is a specified distance. For example, the Affymetrix GeneChip Human Tiling Array 1.0 has 35 base pair resolution. Resolution is measured from the central position of adjacent oligonucleotide probes. For example, 35 pb resolution with 25-mer probes leaves 10 base pair gaps between the oligos. See Data Sheet: GeneChip Human Tiling Arrays PN 702143 Rev. 1 for additional information about tiling arrays. The resolution may be varied, for example, in some aspects the probes may overlap by 1 or more bases, resulting in no gaps between probes. In other aspects the gap may be between 5 and 100 bases on average. For applications of tiling arrays, see, for example, Kapranov et al., Science 296:916 (2002), Kampa et al., Genome Res. 14 :331 (2004) and Cheng et al., Science 308:1149-1154 (2005). Tiling arrays may also be designed to interrogate promoter regions. Such arrays are referred to herein as promoter tiling arrays. Promoter tiling arrays contain probes that are tiled through promoter regions.

ChIP experiments can be used as a powerful tool to complement RNA transcription studies because they enable researchers to study DNA elements that contain modifications, may be proximal to modified histones, or are bound by particular DNA-associating proteins (e.g. transcription factors and polymerases) in vivo. Probe lengths may be, for example, 20-70 bases, in a preferred aspect the probes are 25 bases in length. Large regions of a genome, for example, promoter regions, entire chromosomes or entire genomes can be interrogated using tiling arrays. The design may be unbiased toward annotations, such as characterized genes.

In general, cells are first harvested and fixed with formaldehyde to crosslink DNA to proteins. The cells are then lysed and DNA is sheared into smaller fragments using sonication, followed by immunoprecipitation of the protein-DNA complexes with an antibody directed against the specific protein of interest. Following the immunoprecipitation, crosslinking is reversed, samples are protease-treated to remove proteins, and the purified DNA sample is amplified using a random-prime PCR method to amplify all immunoprecipitated DNA regions. Subsequently, targets are fragmented and labeled to hybridize onto an array, for example, a GeneChip® Tiling Array. Methods for fixing cells, fragmenting chromatin, immunoprecipitation of sheared chromatin, and amplification and labeling of enriched DNA are disclosed.

In a preferred embodiment the assay has a three day workflow. On day 1 cells are fixed to crosslink DNA to protein, sonicated to lyse the cells and shear the chromatin, an aliquot is analyzed to check crosslinking efficiency and the sample is immunoprecipitated using one or more selected antibodies. On day 2 the antibody or antibodies bound to the sample are coupled to a solid support, for example Protein-A-sepharose beads to facilitate washing of the antibody complexes and purification of the DNA that is associated with the antibody and the DNA is decrosslinked and treated with proteinase. On day 3 the immunoprecipitated DNA is cleaned, amplified by PCR, for example, fragmented, labeled and hybridized to arrays. In preferred aspects dUTP is incorporated into the fragments and cleavage and labeling take place in the same reaction.

### EXAMPLES

### Example 1

Each of 4 different TdTs was tested at two different concentrations. The reactions each had 5 μg of single-stranded cDNA from Hela total RNA with 1× NEBuffer 4 and 0.25 mM CoCl₂, 1 μL UDG, 1 μL APE 1 and 1 μL 5 mM DLR in a reaction volume of 50 μl. Differing volumes of the different TdTs were added, 2 and 6 μL of Promega TdT, 4 and 8 μL of Roche TdT, 4 and 8 μl of NEB TdT and 4 and 8 μL of Invitrogen TdT. A 25 μL aliquot of each reaction sample was taken out after 60 minutes at 37° C. and heated at 70° C. for 10 minutes. The remaining 25 μL was incubated for an additional 60 minutes and then heated at 70° C. for 10 minutes. Labeling was assayed using a gel to analyze efficiency of fragmentation and a gel shift assay using NeutraAvidin to determine the efficiency of labeling.

The results indicated that using these reaction conditions the Promega and Roche TdT enzyme solutions were most efficient at fragmentation and labeling. The enzymes from Invitrogen and NEB worked but less effectively.

### Example 2

The Promega TdT was tested using different buffer conditions. Each reaction incuded 5 μg single-stranded cDNA prepared from Hela total RNA, 1 μL UDG, 1 μL APE 1, 1 μL 5 mM DLR and 4 μL Promega TdT in a total reaction volume of 50 μL. The buffer conditions were either 1× promega TdT buffer with 1 mM CoCl₂ or NEBuffer 4 with 1 mM CoCl₂. After 60, 90 or 120 minutes of incubation at 37° C. 10 μL of each reaction was removed and incubated at 70° C. for 10 minutes. Fragmentation and labeling was assayed by gel and gel shift as above.

### Example 3

### Single Step Fragmentation and Labeling of Prokaryotic Sample

A sample of 10 μg of E. coli total RNA was amplified using the prokaryotic amplification protocol (see Affymetrix GeneChip Expression Technical Manual Section 3 P/N 701030 Rev 5). A mixture of dNTP and dUTP was used for 1^{st} strand cDNA synthesis and the single stranded cDNA was cleaned using a column. The uracil containing cDNA was treated either with (1) the standard fragmentation and labeling protocol used for sWTA (separate fragmentation and labeling steps), (2) a one step fragmentation and labeling reaction using NEBuffer 4 and 1 mM CoCl₂ for 60 or 90 minutes or (3) one step fragmentation and labeling using Promega TdT buffer with 1 mM CoCl₂ for 60 or 90 minutes. The samples were hybridized to an E. coli 2.0 GeneChip Array. The results were analyzed to compare percent present calls, call concordance and signal correlation. Both one step fragmentation methods (2) and (3) were comparable to two step methods. The order of performance was NEB 90 minutes>than NEB 60 minutes>Promega 90 minutes>Promega 60 minutes. The NEB buffer at both 90 and 60 minutes performed comparably to the two step method.

### Example 4

### One Step Fragmentation and Labeling on Exon Arrays

Target was prepared using 1 μg total Hela RNA using RiboMinus treatment. The single stranded cDNA was treated with by the standard two step fragmentation and labeling method using Promega TdT, one step using NEBuffer 4 with 1 mM CoCl₂ for 60 minutes at 37 ° C. or one step using NEBuffer 4 with 1 mM CoCl₂ for 90 minutes at 37° C. The products were hybridized to the human all exon array and the hybridization pattern was analyzed for % probes detected above background (DABG), and mean probeset PLIER target response. Both one step fragmentation and labeling methods performed equivalently to the two step method.

### Example 5

### Testing Stability of Functionality of Mixture of APE 1, UDG and TdT

The 3 enzyme mix was formed by mixing 1 μl APE 1 (1,000 U/μl), 1 μl UDG (10 U/μl) and 8 μl TdT (20 U/μl), all from NEB. A control mix of 1 μl APE 1 (1,000 U/μl) and 1 μl UDG (10 U/μl) was also prepared. Target was prepared using 100 ng total Hela RNA following the WTA protocol until single stranded cDNA purification. A first aliquot was treated with the standard two step fragmentation and labeling protocol. A second aliquot was treated by adding the three enzymes individually, a third was treated by adding a three enzyme mix that had been prepared 2 months earlier and stored at −20° C. and a fourth aliquot was treated by adding a mixture of APE 1 and UDG that has been prepared 2 months earlier and stored at −20° C. and TdT. Aliquots 2, 3 and 4 were in NEBuffer 4 plus 1 mM CoCl2, and incubation was for 1.5 hours. All were performed in triplicate and hybridized to an All Exon array for analysis. The % of probes detected above background (averaged over the 3 replicates) was as follows, 52.9 for the addition of the three enzymes individually, 52.4 for the 3 enzyme mix, 50.8 for the 2 enzyme mix and 51.7 for the control. The results indicate that the enzyme mixtures perform nearly the same as adding the enzymes separately and that the mixture can be stored.

### Example 6

### Chromatin Immunoprecipitation and Array Analysis

A. Preparation of Cells

Grow enough cells to allow detection of a single copy gene (usually 5×10⁷ cells, depending on IP efficiency. For each IP use ˜0.5-2×10⁸. For example, grow 200 mL of 1×10⁶ cells/niL for a total of 2×10⁸cells.

B. Cell Fixation, Lysis, and Sonication of Whole Cell Extracts

The protocol may be used with suspension cells or adherent cells. If using adherent cells first harvest cells and resuspend thoroughly in 20 mL of culture media, then treat as suspension cells. Fix cells by adding formaldehyde to a final concentration of 1% (for example, add 5.5 mL of 37% formaldehyde to 200 mL of culture medium). Incubate at room temperature (RT) in fume hood for 10 min, gently swirl 200 mL culture or invert tube containing 20 mL of adherent cells occasionally to mix cells. Add 1/20 volume 2.5 M glycine and incubate RT 5 min with gentle mixing to quench formaldehyde reaction. Perform remaining steps on ice. Pellet cells at 4° C., 1500 rpm (453 g), 4 min and discard supernatant in formaldehyde waste. Wash pellet with 10 mL ice-cold 1× PBS to resuspend cells, and transfer to 15 mL tube. Pellet cells at 4° C., 1500 rpm, discard supernatant and repeat wash. A swing-bucket type rotor may be used. Wash the pellet 3 times with 10 mL Run-on Lysis Buffer. Pellet cells at 1000 rpm (201 g) 5min between washes. Proceed to the next step or flash freeze pellet and store at −80° C.

Resuspend the pellet in ImL MNase reaction buffer+60 μl 100 mM PMSF and bring final reaction volume to 1.5 mL with MNase buffer. Add appropriate units of MNase based on prior optimization of MNase to effectively shear crosslinked chromatin. This can range from 25 U to 200 U or more for each IP performed. Incubate at 37° C., 10 min. Add 30 μl 200 mM EGTA to stop the reaction. Add to the tube: 40 μL 100 mM PMSF, 100 μL 25× protease inhibitor free EDTA tablet, 460 μL MNase reaction buffer, 100 μL 20% SDS, 80 μL 5M NaCl, and 190 μL Nuclease free water for a final sample volume before sonication of 2.5 mL. Sonicate sample to lyse cells and shear DNA to 100-1000 bp fragments. Note: Use optimized shearing conditions. Best sonication conditions were achieved with a Branson Sonifier 450D set at 60% duty, 50% amplitude, 1 min pulses with 1 min rest in an ice bath between pulses, 15 pulses total.

Microcentrifuge 14,000 rpm 10 min at 4° C. to remove cellular debris The sonication efficiency can be checked by taking an aliquot (100 μl) of this supernatant, de-crosslinking it (see below), and running the DNA on an agarose gel. At this point the samples may be divided into aliquots equivalent to ˜5×10⁷ cells

C. Check Sonication Efficiency

Adjust the SDS concentration to 0.5% by adding 100 μL 10 mM Tris pH 8.0 to the 100 μL aliquot taken from the sonicated samples. Add 2 μL Proteinase K and mix well by vortexing. In another aspect Pronase is used in place of Proteinase K. Incubate 42° C. for 2 hr, then 65° C. for 6 hr to overnight (This step can be performed in a thermocycler). Clean-up using Affymetrix cDNA cleanup columns, eluting with 20 μL elution buffer (see protocol below). Load 100-500 ng of purified DNA sample on an agarose gel to check sonication efficiency. Typically, sheared DNA size ranges from 200-4000 bp, with the average size fragment between 500-2000 bp.

D. Immunoprecipate With Specific Antibody

If the sample was frozen, centrifuge again 2000 rpm for 10 min at 4° C. to remove additional precipitates. Transfer supernatant to 15 mL tube and add 4 volumes of IP dilution buffer containing protease inhibitors (tablet from Roche, add before use). Prepare protein A sepharose beads by mixing 50 μl beads with 1 mL IP dilution buffer, pellet 2 min@2000 rpm, repeat, remove all supernatant except ˜100 μL. Pre-clear chromatin by adding 100 μl pre-equilibrated protein A sepharose beads. Incubate on a rotating platform at 4° C. 15 min or longer. Microcentrifuge 2,000 rpm for 2 min. Transfer supernatant to fresh tube and discard beads. Remove 100-300 μl of pre-cleared samples as “input”, store at −20° C. for later use in the protocol. Add 5-10 μg of antibody. In another aspect between 1 and 20 μg of antibody may be used. Incubate on rotating/rocking platform at 4° C. overnight (or for at least 3 hr at RT).

E. Couple to Beads and Wash

Pre-equilibrate protein A sepharose beads: 1 mL IP dilution buffer+100 uL beads for each IP'd sample. Centrifuge 2000 rpm 2 min at 4° C. Discard around 900 μL supernatant: save ˜200 μL of beads in buffer at the bottom of the tube. Transfer 200 μL beads to each sample. Add 40 μL 100 mM PMSF to each tube sample (final conc. 1 mM PMSF in final vol ˜4 mL). Incubate with gentle mixing at 4° C. for 3 hr. Centrifuge at 2000 rpm at 4° C. for 4 min, and then discard supernatant. Resuspend the pellet with 1 mL IP dilution buffer (containing 1 mM PMSF added fresh), mix and transfer to dolphin-nose tube. Centrifuge at 2000 rpm at 4° C. for 2 min and discard supernatant. Repeat step 7 and 8 two more times, and resuspend with 1 mL IP dilution buffer; incubate on rotating mixer 5 min at RT, centrifuge, and discard supernatant. Resuspend the pellet with 700 ul IP dilution buffer (containing 1 mM PMSF), mix, and transfer to spin-X column. Centrifuge at 2000 rpm and discard flow-through. Repeat wash. Wash the beads with 700 μL ChIP wash 1. Incubate on rocking mixer for 1 min at RT. Centrifuge at 2000 rpm at RT and discard flow-through. Wash the beads with 700 μL ChIP wash 2. Incubate on rocking mixer for 5 min at RT. Centrifuge at 2000 rpm at RT and discard flow-through. Wash the beads with 700 μL ChIP wash 3. Incubate on rocking mixer for 5 min at RT. Centrifuge at 2000 rpm at RT and discard flow-through. Wash the beads with 700 μL 1× TE. Incubate on rocking mixer for 1 min at RT. Centrifuge at 2000 rpm at RT and discard flow-through. Repeat steps 22 through 24 Transfer the spin-X column with beads to a new dolphin-nose tube. Add 200 μL Elution buffer to the column. Incubate at 65° C. for 30 min. Centrifuge at 3000 rpm for 2 min at RT. Add 100 μL Elution buffer to the column. Centrifuge at 3000 rpm for 2 min at RT. This 300 μL eluted sample is referred to herein as the “enriched” or “IP'd” sample. If using the Input sample as the control (from step D8), it is preferably included in subsequent steps.

F. Reverse Crosslinks

Take out saved input sample (from step D8) from −20° C. Add 20% SDS to Input sample to make the final concentration to 0.5% SDS. Add 30 μL Proteinase K (20 mg/mL) to each IP and Input sample: final concentration=2 μg/μL in 300 μL, mix well. Incubate at 65° C. overnight.

G. Cleanup De-crosslinked Samples

Clean up samples using Affymetrix cDNA cleanup columns. Elute with 2× 20 μL elution buffer.

H. PCR Amplification of Immunoprecipitated DNA Targets

Use 50% of IP'd or 20 ng input DNA for initial round of linear amplification. Adjust sample volume to 37 μL containing required DNA amounts. Set up first round reaction by mixing for each reaction, 37 μL Purified DNA, 12 μL 5× sequenase buffer and 4 μL Primer A (40 μM). Primer A: GTTTCCCAGTCACGATCNNNNNNNNN (SEQ ID NO. 1). Cycle conditions: are 94° C. for 4 min, place the samples on ice and set themocycler to 10° C. hold while preparing and adding first cocktail to each reaction (7.5 μL). The cocktail is made by mixing 0.5 μL 10 mg/ml BSA, 3 μL 0.1 M DTT, 2.5 μL10 mM dNTPs and 1.5 μL diluted sequenase (1/10 from 13 U/μl stock) for each reaction. Mix well by pipetting, and put the samples back in thermocycler block. Incubate at 10° C. for 5 min, Ramp from 10° C. to 37° C., 37° C. for 8 min, 94° C. for 4 min, Place the samples on ice, Set themocycler to 10° C. hold, Add 1.5 μL of 1.3 U/μL sequenase to each sample, Put the samples back in the thermocycler 10° C. for 5 min, Ramp from 10° C. to 37° C., 37° C. for 8 min and 4° C. hold. Upon completion of first round, purify with Affymetrix cDNA cleanup columns, eluting with 2× 20 μL of elution buffer. Set up the PCR Reaction by mixing 36 μL “Round A” DNA from above 10 μL10× PCR buffer 2 μL25 mM MgCl22.5 μL10 mM dNTPs+dUTP0.8 μL100 μM Primer B, 2 μL 5U/μl Taq and 46.7 μL Nuclease-free water for each reaction. Primer B is GTTTCCCAGTCACGATC (SEQ ID NO. 2). Cycle conditions are 95° C. for 2 min, 94° C. for 30 sec, 40° C. 30 sec, 50° C. 30 sec, 72° C. 1 min, Repeat b)-e) for 34 additional cycles and 4° C. hold. Check amplified DNA on 1% agarose gel. Purify PCR samples with Affymetrix cDNA cleanup columns, eluting with 2× 20 μL of elution buffer and measure DNA using Nanodrop or other UV spectrophotometer.

I. Fragmentation of Amplified Targets

Fragment the samples by mixing the following reagents for each reaction: 7.5 μg Double-Stranded DNA, 4.8 μL 10× Fragmentation Buffer, 1.5 μL 10 U/μL UDG, 2.25 μL 100 U/μL APE 1, and RNase-free Water up to 48 μL total reaction volume. Add the above mix to the samples, flick-mix, and spin down the tubes. Incubate the reactions at: 37° C. for 1 hour, 93° C. for 2 minutes and 4° C. for at least 2 min. Flick-mix, spin down the tubes, and transfer 45 μL of the sample to a new tube. The remainder of the sample is to be used for fragmentation analysis using a Bioanalyzer or agarose gel. Please see the Reagent Kit Guide that comes with the DNA 1000 LabChip Kit for instructions. If not labeling the samples immediately, store the fragmented Double-Stranded DNA at −20° C.

J. Labeling of Fragmented Double-Stranded DNA:

Prepare the labeling reactions by mixing the following for each reaction: 45 μL Fragmented Double-Stranded DNA, 12 μL 5× TdT Buffer, 2 μL TdT and 1 μL 5 mM DNA Labeling Reagent. Total volume is 60 μL. Add 15 μL of the Double-Stranded DNA Fragmentation Mix to the DNA samples, flick-mix, and spin them down. Incubate the reactions at: 37° C. for 60 min. then 70° C. for 10 minutes and 4° C. for at least 2 min. Remove 4 μL of each sample for Gel-shift analysis (optional). In a preferred aspect, steps B-D may be performed on Day 1, steps E and F on Day 2 and steps G-J on Day 3.

An exemplaray protocol and workflow for hybridizing the products to an array is disclosed below:

A. Hybridization of Labeled Target on the Arrays

Prepare the Hybridization Cocktail in a 1.5 mL RNase-free microfuge tube as follows (volumes given for a single reaction followed by final concentration or amount). Fragmented and labeled DNA Target, ˜60.0 μL (if a portion of the sample is set aside for gel shift analysis this volume is 56 μL) for ˜7.5 μg, control oligonucleotide B2 4.2 μL for 50 pM, 20× Eukaryotic hybridization contyrols (bioB, bioC, bioD, cre) 12.5 μL for 1.5, 5, 25 and 100 pM, respectively, herring sperm DNA (10 mg/mL) 2.5 μL for 0.1 mg/mL, Acetylated BSA (50 mg/mL) 2.5 μL for 0.5 mg/mL, 2× Hybridization Buffer 125 μL for 1×, DMSO 17.5 μL for 7%, RNase free H₂O up to 250.0 μL.

Flick-mix, and centrifuge the tube. Heat the Hybridization Cocktail at 99° C. for 5 min. Cool to 45° C. for 5 minutes, and centrifuge at maximum speed for 1 minute. Inject ˜200 μL of the specific sample into the array through one of the septa. Save the remaining hybridization cocktail in −20° C. for future use. Place array in 45° C. hybridization oven, at 60 rpm, and incubate for 16 hr.

B. Array Wash, Stain, and Scan

Use the fluidics protocol FS450_{—}0001 for wash and stain if using an FS450 fluidics station, or alternatively, if using an FS400, use the EukGE-WS2v5 protocol and add Array Holding Buffer to the cartridge manually prior to scanning. Scan the probe array according to the GeneChip Expression Analysis Technical Manual (Section 2: Eukaryotic Sample and Array Processing). In many aspects the step of cleanup of Double-Stranded DNA is preformed using the GeneChip Sample Cleanup Module according to the following procedure: If not already done, add 24 mL of Ethanol (100%) to the cDNA Wash Buffer supplied in the GeneChip Sample Cleanup Module. Add 5× volume of cDNA Binding Buffer to sample, and vortex for 3 seconds. Apply the sample to a cDNA Spin Column sitting in a 2 mL Collection Tube (max capacity of column=700 μL; if volume exceeds 700 μL, spin 700 μL at>8,00033 g for 1 min, discard flow-through, and repeat). Spin at >8,000×g for 1 minute. Discard the flow-through. Transfer the cDNA Spin Column to a new 2 mL Collection Tube and add 750 μL of cDNA Wash Buffer to the column. Spin at >8,000×g for 1 minute and discard the flow-through. Open cap of the cDNA Spin Column, and spin at<25,000×g for 5 minutes with the caps open. Discard the flow-through, and place the column in a 1.5 mL collection tube. Pipette recommended amount of cDNA Elution Buffer directly to the column membrane and incubate at room temperature for 1 minute. Then, spin at<25,000×g for 1 minute. Take 2 μL from each sample to determine the yield by spectrophotometric UV measurement at 260nm, 280 nm and 320 nm. The following formula may be used: Concentration of Double-Stranded cDNA (μg/μL)=[A₂₆₀-A₃₂₀] ×0.05× dilution factor.

The following buffers may be used in preferred embodiments: Run on Lysis Buffer (Store at 4° C.) is 10 mM Tris-HCl pH 7.5, 10 mM NaCl, 3 mM MgCl₂, 0.5% NP-40 and 1 mM PMSF (added fresh). MNase Buffer (Store at RT) is 10 mM Tris-HCl pH 7.5, 10 mM NaCl, 3 mM MgCl₂, 1 mM CaCl₂, 4% NP-40 and 1 mM PMSF (add fresh). IP Dilution Buffer (Store at RT without protease inhibitors) is 20 mM Tris-HCl pH 8, 2 mM EDTA, 1% Triton X-100, 150 mM NaCl and Protease inhibitors (tablet/Roche). ChIP Wash 1 (Store at RT) is 20 mM Tris-HCl pH 8, 2 mM EDTA, 1% Triton X-100, 150 mM NaCl and 1 mM PMSF (add fresh). ChIP Wash 2 (Store at RT) is 20 mM Tris-HCl pH 8, 2 mM EDTA, 1% Triton X-100, 0.1% SDS, 500 mM NaCl and 1 mM PMSF (add fresh). ChIP Wash 3 (Store at RT) is 10 mM Tris-HCl pH 8, 1 mM EDTA, 0.25M LiCl, 0.5% NP-40, 0.5% deoxycholate (use sodium salt, Sigma D-6750). Elution Buffer is 25 mM Tris-HCl pH 7.5, 5 M EDTA and 0.5% SDS. Holding Buffer is 1× Array Holding Buffer (Final 1× concentration is 100 mM MES, 1M [Na+], 0.01% Tween-20). For 100 mL mix 8.3 mL of 12× MES Stock Buffer, 18.5 mL of 5M NaCl, 0.1 mL of 10% Tween-20, and 73.1 mL of water and Store at 2° C. to 8° C., and shield from light.

### CONCLUSION

All cited patents, patent publications and references are incorporated herein by reference for all purposes. It is to be understood that the above description is intended to be illustrative and not restrictive. Many variations of the invention will be apparent to those of skill in the art upon reviewing the above description. The scope of the invention should, therefore, be determined not with reference to the above description, but should instead be determined with reference to the appended claims, along with the full scope of equivalents to which such claims are entitled.

What is claimed is:

1. A method for obtaining a nucleic acid amplification product comprising labeled cDNA fragments from a nucleic acid sample containing RNA, the method comprising:
a) providing a first nucleic acid sample comprising RNA;
b) amplifying the first nucleic acid sample to obtain a second nucleic acid sample comprising cDNA, wherein said cDNA contains uracil by a method comprising the steps of (i) synthesizing first strand cDNA from said RNA by reverse transcription using primers comprising a random portion and an RNA polymerase promoter portion;
(ii) synthesizing second strand cDNA to obtain double stranded cDNA comprising an RNA polymerase promoter;
(iii) generating cRNA by in vitro transcription of said double stranded cDNA; and
(iv) generating cDNA from said cRNA by reverse transcription using random primers in the presence of dUTP followed by removal of the cRNA strand by a method selected from the group consisting of RNase H treatment and alkali treatment; and,
c) fragmenting the double stranded cDNA and labeling the resulting fragments, wherein the fragmenting and labeling take place in a single reaction, by a method comprising incubating the double stranded cDNA in a reaction comprising UDG, an AP endonuclease, TdT and a labeled nucleotide to generate labeled cDNA fragments.

2. The method of claim 1 wherein the AP endonuclease is APE 1.

3. The method of claim 2 wherein the APE 1, UDG and TdT are mixed to form an enzyme mixture and an aliquot of the enzyme mixture is added to the reaction in step c).

4. The method of claim 1 wherein the volume of the reaction of step c) is between 35 and 60 microliters.

5. The method of claim 1, wherein said uracil containing cDNA is obtained by reverse transcribing cRNA in the presence of a first amount of dTTP and a second amount of dUTP, wherein the ratio of dTTP to dUTP is between 3 to 1 and 8 to 1.

6. The method of claim 1, wherein the average size of the labeled cDNA fragments is about 40 to 150 bases in length.

7. The method of claim 1, wherein the average size of the labeled cDNA fragments is 40 to 70 bases in length.

8. The method of claim 1 wherein the reaction in step c) contains between 0.25 and 1 mM CoCl₂.

9. A method of determining the expression level of a plurality of RNAs in a nucleic acid sample said method comprising:
synthesizing first strand cDNA from said RNAs by reverse transcription using primers comprising a random portion and an RNA polymerase promoter portion;
synthesizing second strand cDNA to obtain double stranded cDNA comprising an RNA polymerase promoter;
generating cRNA by in vitro transcription of said double stranded cDNA; and
generating cDNA from said cRNA by reverse transcription using random primers in the presence of dUTP followed by removal of the cRNA strand by a method selected from the group consisting of RNase H treatment and alkali treatment;
cleaving and fragmenting the cDNA by a method comprising incubating the cDNA in a fragmentation and labeling reaction wherein the reaction comprises UDG, an AP endonuclease and TdT, to generate labeled cDNA fragments;
hybridizing said labeled cDNA fragments to an array of probes to generate a hybridization pattern; and
analyzing the hybridization pattern to determine the expression level of a plurality of RNAs in the sample.

10. The method of claim 9 wherein the AP endonuclease is APE 1.

11. The method of claim 10 wherein the UDG, APE 1 and TdT are first mixed to form a pre-mix and then an aliquot of the pre-mix is added to the fragmentation and labeling reaction.

12. A kit comprising an enzyme mixture of APE 1, UDG and TdT in a single tube.

13. The kit of claim 12 further comprising a buffer, a solution of CoCl₂ and a solution of DLR.

14. The kit of claim 13 wherein the buffer is a concentrated solution of Tris-acetate, potassium acetate, magnesium acetate and ditiothreitol with a pH of about 7.9 at 25 ° C.

15. The kit of claim 13 wherein the enzyme mixture comprises at least 0.3% detergent.

16. The kit of claim 13 further comprising a solution comprising a labeled nucleotide or nucleotide analog.

17. A method for identifying a plurality of regions of nucleic acid, wherein said regions are in physical proximity to a nucleic acid binding protein, said method comprising:
a) obtaining a suspension of cells;
b) fixing said cells by (i) adding formaldehyde to said suspension, (ii) incubating for a period of time and (iii) stopping the fixing reaction;
c) washing the fixed cells;
d) disrupting the cells and sheering the nucleic acid;
e) immunoprecipitating protein-nucleic acid complexes using an antibody to a nucleic acid binding protein of interest;
f) recovering nucleic acid from the immunoprecipitated complexes obtained in (e);
g) performing a linear amplification step on the nucleic acids recovered in (f), wherein said linear amplification step comprises extension of a primer comprising a 3′ random portion and a 5′ constant portion;
h) amplifying the products of (g) by PCR with a primer that comprises at least 15 contiguous bases of said constant portion and wherein said amplification is done in the presence of dUTP to generate dUTP containing amplified fragments;
i) fragmenting and labeling the amplified fragments in a reaction comprising a uracil DNA glycosylase, an AP endonuclease, a terminal deoxynucleotidyl transferase and a biotin labeled nucleotide to obtain labeled fragments;
j) hybridizing the labeled fragments to an array of oligonucleotides arranged in features of the array and wherein features of the array become labeled as a result of hybridization and wherein a pattern of labeled features is obtained; and
j) analyzing the pattern to identify regions of the nucleic acid that are associated with said protein of interest.

18. The method of claim 17 wherein said AP endonuclease is APE .

19. The method of claim 17 wherein said array is a tiling array comprising more than 1 million probes spaced at a resolution of 30 to 35 bases.

20. The method of claim 17 wherein said array is a promoter tiling array.
